(19) United States
(12) Patent Application Publication
(10) Pub. No.: US 2001/0028875 A1
(43) Pub. Date: Oct. 11, 2001

(54) Screening methods for gram-positive enterococcal virulence factors

(76) Inventors: Frederick M. Ausubel, Newton, MA (US); Stephen B. Calderwood, Wellesley, MA (US); Danielle Garsin, Brookline, MA (US); Costi D. Sifri, Quincy, MA (US)

Correspondence Address: CLARK & ELBING LLP, 176 FEDERAL STREET, BOSTON, MA, 02110-2214, US

(21) Appl. No.: 09/791,318

(22) Filed: Feb. 23, 2001

Related U.S. Application Data
(60) Provisional application No. 60/185,073, filed on Feb. 25, 2000.

(51) Int. Cl. A61K 49/00

(52) U.S. Cl. 424/9.2

(57) ABSTRACT

Methods are provided for identifying Enterococcal virulence factors. Methods are also provided for screening compounds that inhibit pathogenicity of an Enterococcal pathogen.

### BACKGROUND OF THE INVENTION

This application claims benefit of U.S. Provisional application 60/185,073 filed on Feb. 25, 2000.

The invention relates to screening methods for identifying pathogen virulence factors and for identifying drugs that inhibit pathogen infections.

The gram-positive pathogens in the genus Enterococcus are an increasingly problematic source of nosocomial infections, in part due to multi-drug resistance. Enterococcus can cause diseases such as bacteremia and endocarditis. These pathogens can also infect the urinary tract and skin wounds in immunocompromised individuals. Infection can be fatal if the bacteria cannot be neutralized.

Despite their increasing prevalence as infectious agents, little is known about how these bacteria cause disease. Only cytolysin and aggregation substance have been studied rigorously enough to be established as virulence factors in mammalian models of Enterococcus faecalis pathogenesis. Other virulence factors, such as certain proteases, are believed to contribute to pathogenesis, but have not been studied adequately in mammalian model systems.

One reason that little is known about Enterococcal virulence factors is that the model systems used to study these bacteria, the favorite being a rabbit model of endocarditis, are expensive and unwieldy. Using a mammalian model system to screen for these virulence factors would be virtually impossible. Accordingly, there exists a need for straightforward, inexpensive, and reliable methods to identify Enterococcal virulence factors. Also needed, are easy, accurate screening methods that would greatly simplify the drug discovery process aimed at identifying molecules that inhibit Enterococcal pathogenicity or promote host resistance to this pathogen.

### SUMMARY OF THE INVENTION

The invention provides a novel approach to identifying Enterococcal virulence factors and for identifying compounds for treating bacterial pathogenesis.

In a first aspect, the invention features a method for identifying an Enterococcal virulence factor. The method, in general, involves the steps of: (a) exposing a nematode to a mutagenized Enterococcal pathogen; (b) determining whether the Enterococcal mutant infects the nematode, a reduction of disease in the nematode relative to that caused by the non-mutagenized Enterococcal pathogen indicating a mutation in an Enterococcal virulence factor; and (c) using the mutation as a marker for identifying the Enterococcal virulence factor. In preferred embodiments, the Enterococcal pathogen is Enterococcus faecalis (e.g., Enterococcus faecalis strain V583) and the nematode is Caenorhabditis elegans (e.g., a wild type or mutant worm). In other preferred embodiments, the method utilizes an Enterococcal/C. elegans killing assay.

In a second aspect, the invention features a method of identifying a compound that inhibits pathogenicity of an Enterococcal pathogen. The method, in general, involves the steps of: (a) providing a nematode infected with an Enterococcal pathogen; (b) contacting the infected nematode with a test compound; and (c) determining whether the test compound inhibits the pathogenicity of the Enterococcal pathogen in the nematode. In preferred embodiments, the Enterococcal pathogen is Enterococcus faecalis (e.g., Enterococcus faecalis strain V583) and the nematode is Caenorhabditis elegans (e.g., a wild type or mutant worm). Preferably, the test compound is provided in a compound library. In other preferred embodiments the test compound is a small organic compound; or is a peptide, peptidomimetic, or antibody or fragment thereof. In still other preferred embodiments, the inhibition of pathogenicity is measured by an Enterococcal/C. elegans killing assay.

In yet another aspect, the invention features an isolated nematode (e.g., Caenorhabditis elegans), that includes an isolated Enterococcal pathogen. In preferred embodiments, the Enterococcal pathogen is Enterococcus faecalis, Enterococcus faecalis strain V583, Enterococcus faecium, or is a mutated Enterococcal pathogen.

By “virulence factor” is meant a cellular component (e.g., a protein such as a transcription factor or a molecule) without which a pathogen is incapable of causing disease or infection in a eukaryotic host organism (e.g., a nematode or mammal). Such components are involved in the adaptation of the bacteria to a host (e.g., a nematode host), establishment of a bacterial infection, maintenance of a bacterial infection, and generation of the damaging effects of the infection to the host organism. Further, the phrase includes components that act directly on host tissue, as well as components which regulate the activity or production of other pathogenesis factors.

By “infection” or “infected” is meant an invasion or colonization of a host animal (e.g., nematode) by pathogenic bacteria that is damaging to the host.

By “inhibits pathogenicity of an Enterococcal pathogen” is meant the ability of a test compound to decrease, suppress, attenuate, diminish, arrest, or stabilize the development or progression of an Enterococcal-mediated disease or infection in a eukaryotic host organism. Preferably, such inhibition decreases pathogenicity by at least 5%, more preferably by at least 25%, and most preferably by at least 50% or more, as compared to symptoms in the absence of the test compound in any appropriate pathogenicity assay (for example, those assays described herein). In one particular example, inhibition may be measured by monitoring pathogenic symptoms in a nematode infected with an Enterococcal pathogen exposed to a test compound or extract, a decrease in the level of pathogenic symptoms relative to the level of symptoms in the host organism not exposed to the compound indicating compound-mediated inhibition of the Enterococcal pathogen.

The present invention provides a number of advantages. For example, the invention facilitates the identification of novel targets and therapeutic approaches for preparing therapeutic agents active on Enterococcal virulence factors and genes. The invention also provides long awaited advantages over a wide variety of standard screening methods used for distinguishing and evaluating the efficacy of a compound against Enterococcal pathogens. In one particular example, the screening methods described herein allow for the simultaneous evaluation of host toxicity as well as anti-Enterococcal potency in a simple in vivo screen. Moreover, the methods of the invention allow one to evaluate the ability of a compound to inhibit Enterococcal pathogenesis, and, at the same time, to evaluate the ability of the compound to stimulate and strengthen a host's response to Enterococcal pathogenic attack.

Accordingly, the methods of the invention provide a straightforward means to identify compounds that are both safe for use in eukaryotic host organisms (i.e., compounds which do not adversely affect the normal development and physiology of the organism) and efficacious against Enterococcal pathogenic microbes. In addition, the methods of the invention provide a route for analyzing virtually any number of compounds for anti-Enterococcal pathogenic effect with high-volume throughput, high sensitivity, and low complexity. The methods are also relatively inexpensive to perform and enable the analysis of small quantities of active substances found in either purified or crude extract form. Furthermore, the methods disclosed herein provide a means for identifying anti-pathogenic compounds which have the capability of crossing eukaryotic cell membranes and which maintain therapeutic efficacy in an in vivo method of administration. In addition, the above-described methods of screening are suitable for both known and unknown compounds and compound libraries.

Other features and advantages of the invention will be apparent from the following description of the preferred embodiments thereof, and from the claims.

### DETAILED DESCRIPTION

The drawings will first be described.

### DRAWINGS

FIG. 1 shows C. elegans killing by Enterococcal clinical isolates.

FIG. 2 shows C. elegans killing by Enterococcus strains E002, E006, and V583.

FIG. 3 shows C. elegans killing by Enterococcus strains V583, OG1, OG1 (pAD1), OG1 (pCF10), and E007.

FIG. 4 shows C. elegans killing by Enterococcus strains containing plasmids pAD1 or pAD1-cylΔ.

Below we describe experimental evidence demonstrating that Enterococcus causes disease in the nematode C. elegans, and that C. elegans feeding on lawns of Enterococcus faecalis die over the course of a few days as a result of a pathogenic process. Data is also presented demonstrating that at least one known E. faecalis virulence factor, cytolysin, required for maximum virulence in mammalian models, enhances the killing of C. elegans, validating the use of a C. elegans host as a model for mammalian pathogenesis. The Enterococcal/C. elegans killing assay described herein therefore provides a useful system for identifying novel Enterococcal virulence factors, as well as for identifying compounds that either inhibit Enterococcal pathogenicity, promote a host's resistance to the pathogen, or both. The following experimental examples are intended to illustrate, not limit, the scope of the claimed invention.

### C. elegans/Enterococcus Killing Assays

To monitor Enterococcus-mediated killing, C. elegans assays were carried out as follows. Brain heart infusion (BHI) agar medium (Difco) was autoclaved and poured into 35 mm tissue culture plates (Fisher). Appropriate antibiotics were added to the medium before pouring that prevented growth of E. coli, but allowed growth of the particular Enterococcus strains being tested. For strains E001, E002, E003, E006 and E009, 12.5 μg/ml tetracycline was used. For strain V583, 200 μg/ml gentamycin was used. For strain E007, 50 μg/ml ampicillin was used. For strains OG1 and OG1 (pAD1), 250 μg/ml of spectinomycin was used. For strain OG1 (pCF10), 250 μg/ml spectinomycin and 12.5 μg/ml tetracycline were used. For strains FA2-2, FA2-2 (pAM714), and FA2-2 (pAM771), 50 μg/ml gentamycin were used.

Bacterial lawns of Enterococcus were prepared as follows. On the tissue culture plate, 2 ml of BHI was inoculated with a single colony of the appropriate strain, grown at 37° C. for 4 to 5 hours, and 10 μl of the culture was plated on each plate. The plates were incubated at 37° C. overnight, and then brought to room temperature for 2 to 5 hours. Thirty C. elegans, at the L4 larval stage, were then placed on the lawn from a plate of OP50 E. coli. The plates were incubated at 25° C., and the number of worms found dead compared to the total number of plated worms was counted at approximately 24 hour intervals. Each experimental condition in the following experiments was done in triplicate and repeated at least twice.

### C. elegans Killing By Enterococcal Clinical Isolates

Six different strains of Enterococcus were obtained from the clinical microbiology laboratory at Massachusetts General Hospital (Boston, Mass.) and were designated E001, E002, E003, E006, E007, and E009. Standard clinical methods were used to identify strains E003 and E007 as Enterococcus faecium, and strains E001, E002, E006, and E009 as Enterococcus faecalis. In general, Enterococcus faecalis causes approximately 80-90% of the Enterococcal infections in humans, and Enterococcus faecium causes approximately 10-20%.

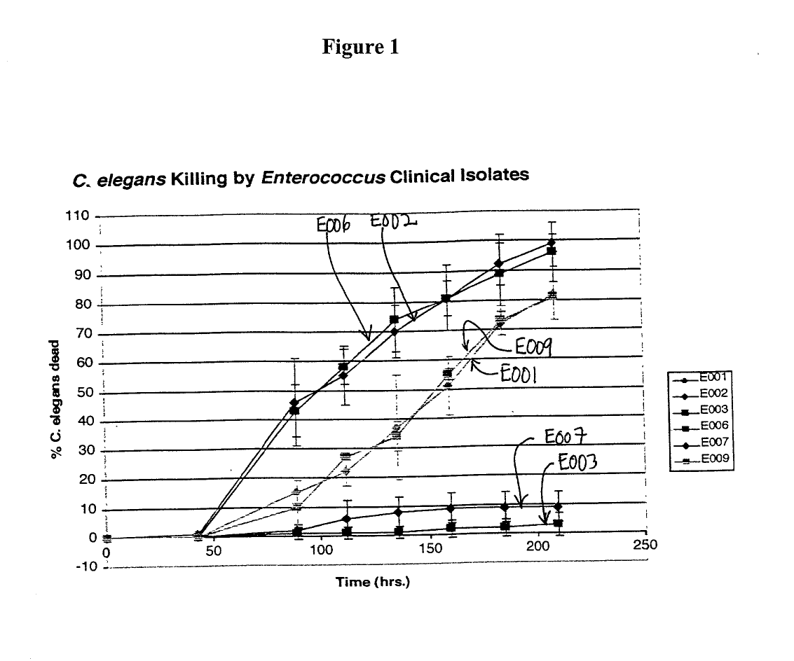

Using the above-described killing assay protocol, the percentage of C. elegans dead as a function of time feeding on each of the Enterococcus strains was determined. As shown in FIG. 1, clinical isolates E002 and E006 were found to kill C. elegans most quickly, with a LT₅₀ of about 100 hours. E001 and E009 killed more slowly, with a LT₅₀ of about 150 hours. E003 and E007 did not cause any significant killing of C. elegans. These data suggest that Enterococcus faecalis, but not Enterococcus faecium, can kill C. elegans. Also, the differences between different strains of Enterococcus faecalis suggested that there might be identifiable genetic differences that caused the observed range in killing efficiency.

### C. elegans Killing By Enterococcus faecalis strain V583

Strain V583 is a vancomycin-resistant clinical isolate of E. faecalis. It was first described in the late 1980's when vancomycin resistance emerged as a problem among Enterococcal infections. The genome of E. faecalis strain V583 is currently being sequenced by TIGR (The Institute for Genomic Research). The sequence is publicly available at (http://ftp.tigr.org/tdb/mdb/mdb.html).

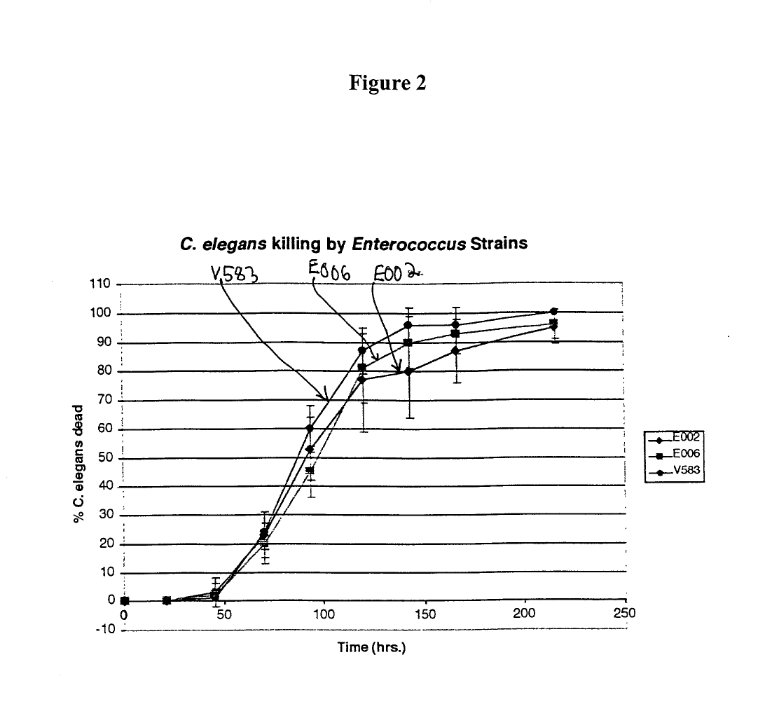

FIG. 2 shows the killing of C. elegans by strains E002, E006, and V583. In comparison to E002 and E006, E. faecalis strain V583 was found to kill just as effectively with a LT₅₀ of about 100 hours (FIG. 2).

### C. elegans Killing By Isogenic Enterococcus faecalis Expressing Different Known Virulence Factors

E. faecalis contains a number of naturally occurring conjugative plasmids. Two such plasmids, called pADi (Jett et al., Clinical Microbiol. Rev. 7: 462-478, 1994) and pCF10 (Leonard et al., Proc. Natl. Acad. Sci. 93: 260-264, 1996) are well studied. A strain that does not have a particular plasmid (recipient) releases a peptide signal called a pheromone which, in turn, causes a strain that does contain the plasmid (donor) to produce aggregation substance (AS) on its surface. AS binds to Enterococcus binding substance (EBS) on the recipient, forming a mating aggregate which allows a copy of the plasmid to be conjugatively transferred from the donor to the recipient.

In addition to its role in plasmid conjugation, AS is also thought to play a role in pathogenesis by helping E. faecalis bind to host tissues. For example, strains producing AS bound more tightly to pig renal tubular cells than strains that did not produce AS. The genes for producing AS are located on both pADI and pCF10.

In addition to AS, cytolysin (Cy1) is another virulence factor that is capable of lysing both eukaryotic and other prokaryotic cells. The cytolysin operon is present on pAD1, but not pCF10. When both AS and Cy1 are expressed in rabbit endocarditis models, there is a significant increase in mortality.

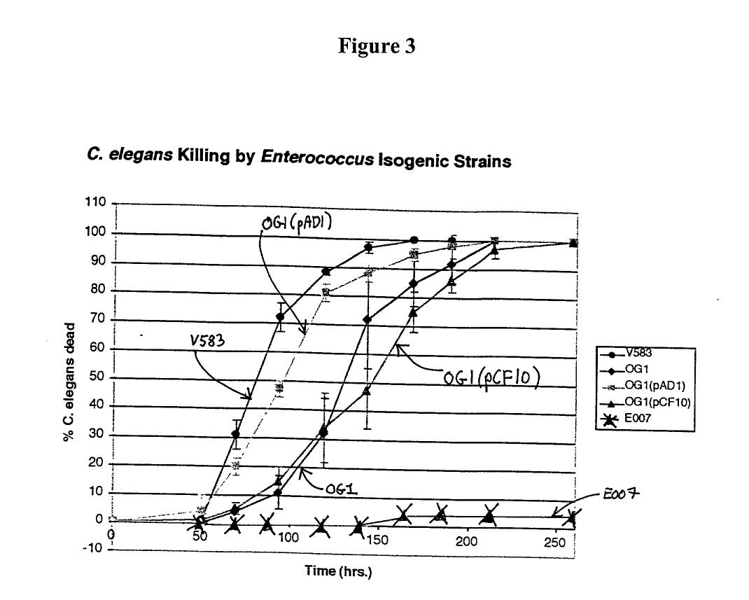

To examine the role of AS and Cy1 in nematode infection, isogenic strains of E. faecalis that were plasmid-free (strain OG1), harbored a plasmid containing genes for both AS and Cy1 (strain OG1 containing pAD1), or harbored a plasmid containing only the gene for AS (strain OG1 containing pCF10) were examined in the above-described killing assay. E. faecalis strains V583 and E007 were used as controls. Results of these experiments are shown in FIG. 3.

Strain OG1 containing pAD1 was found to kill C. elegans significantly faster than strain OG1 or strain OG1 with pCF10. These results appear to indicate that cytolysin contributes to pathogenesis in C. elegans, but that aggregation substance has little or no effect.

### C. elegans Killing By Isogenic Enterococcus Strains Containing Conjugative Plasmids: pAD1 vs. pAD1-cylΔ

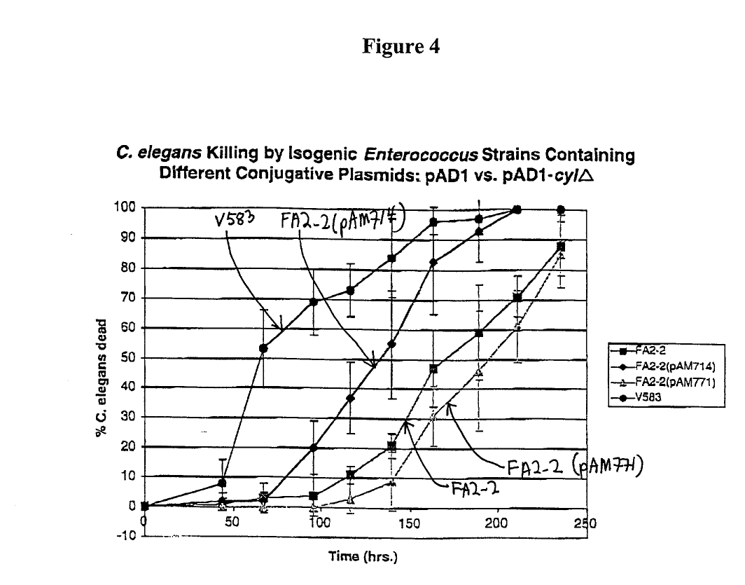

To examine whether the virulence factor, cytolysin, is responsible for the faster killing of the C. elegans, the killing rates of isogenic E. faecalis strains containing different mutations in pAD1 (Ike et al., J. Bacteriol. 172: 155-163, 1990) were examined. As shown in FIG. 4, the strain containing wild type pAD1 (FA2-2 containing pAM714) was found to kill C. elegans faster than the plasmid-free strain or the strain containing pAD1 with a deletion in the promoter of the cytolysin operon (FA2-2 containing pAM771). These data indicated that the virulence factor, cytolysin, caused faster killing of C. elegans.

To summarize, we have developed a new pathogen/host model system employing E. faecalis and C. elegans. We have shown that different strains of E. faecalis kill C. elegans at different rates, and that E. faecium does not cause significant mortality. The sequenced strain of E. faecalis kills very effectively making it an ideal choice for mutagenesis studies. The known mammalian virulence factor, cytolysin, was also found to increase the rate of killing, suggesting that C. elegans is a valid model host for studying mammalian pathogenesis by E. faecalis. This model system provides a potentially valuable tool for identifying novel E. faecalis virulence factors, and for developing a better understanding of this problematic pathogen.

### Nematode Screening Systems For Identifying Enteroccocal Virulence Factors

Based on the results described above showing that E. faecalis virulence factor is involved in pathogenicity of C. elegans, we have developed a method for identifying virulence determinants important for pathogenicity of Enterococcus. The screen, in general, utilizes the above-described Enterococcal/nematode killing assays and exploits the ability to readily screen thousands of randomly generated Enterococcal mutants. In addition to using wild type host worms in the killing assays, mutants that are constipated or defecation defective, such as aex-2 and unc-25, mutants that are grinding defective, such as phm-2 and eat-14, and specific ABC transporter mutants such as pgp-4 and mrp-1 may be utilized as well.

In general, a strain of Enterococcus is mutated according to standard methods known in the art and then subsequently evaluated for its ability to induce disease in the nematode host organism. A mutagenized pathogen found to have diminished pathogenicity or which is rendered non-pathogenic is useful in the method of the invention. Such mutant pathogens are then used for identifying host-dependent or host-independent virulence factors responsible for pathogenicity according to methods known in the art.

The following is a working example of a virulence factor nematode screening system which utilizes the human clinical isolate E. faecalis strain V583 found to be infectious in the C. elegans nematode feeding model. Strain V583 is a vancomycin-resistant variety of E. faecalis, which contains plasmids amounting to an estimated 100 kb. It contains a set of seven genes spanning about 7 kb which contribute to its resistance to the vancomycin antiobiotic. The advantage of using a nematode as a host for studying this mammalian pathogen is the relative simplicity of identifying non-pathogenic Enterococcus mutants in the nematode.

In one preferred working example, in which survival is monitored, four to eight C. elegans worms (e.g., L4 larvae) are placed on a lawn of mutagenized E. faecalis strain V583, and survival is monitored after approximately one hundred to two hundred hours according to the methods described herein. An Enterococcus pathogen, such as E. faecalis strain V583, is mutated according to any standard procedure, e.g., standard in vivo or in vitro insertional/transponson mutagenesis methods (see, e.g., Ike et al., J. Bacteriol. 172:155-63, 1990; Munkenbeck et al., Plasmid 24: 57-67, 1990; Kleckner et al., J. Mol. Biol. 116: 125, 1977). Other methods are also available, e.g., chemical mutagenesis, or directed mutagenesis of DNA. After approximately one hundred to one hundred fifty hours, very few or no live worms are found on a plate seeded with wild-type, pathogenic E. faecalis strain V583, whereas on a plate with mutagenized E. faecalis strain V583, increased survival (e.g., as determined by an increased LT₅₀) of the worms is observed. Thus, the ability of worms to grow in the presence of mutated E. faecalis strain V583 is an indication that a gene responsible for pathogenicity has been inactivated. The positions of the inactivating mutations are then identified using standard methods, (e.g., by polymerase chain reaction and sequencing of insertion/transposon junctions or by mapping), leading to the cloning and identification of the mutated virulence factor(s) (e.g., by nucleotide sequencing).

In another working example, in which survival and reproduction is monitored, two C. elegans worms (e.g., L4 hermaphrodite larvae) are placed on a lawn of mutagenized E. faecalis strain V583, and worm progeny is monitored. Strain V583 is mutated according to standard methods. After approximately one hundred to one hundred fifty hours, very few or no live worms are found on a plate seeded with wild-type, pathogenic E. faecalis strain V583, whereas on a plate with the V583 mutant, hundreds or thousands of live progeny of the initial two hermaphrodite worms are present. Thus, the ability of worms to grow and reproduce in the presence of mutated V583 is taken as an indication that a gene responsible for pathogenicity has been inactivated. The mutated virulence factor is then identified using standard methods.

### Mouse Pathogenicity Screening Assays

To further evaluate the virulence of Enterococcal mutants identified in the above-described nematode screening assays, mouse pathogenicity/mortality studies are performed as follows. Female ICR Mice (Taconic, Germantown, N.Y. or Charles River, Wilmington, Mass.) weighing 20 to 30 grams and housed 5 per cage, are used for evaluating the virulence of Enterococcal mutants. Mice, in groups of 6-10, are injected intraperitoneally with mutant bacteria in sterile rat fecal extracts (SRFE) as described below. The survival of mice receiving mutant bacteria is then compared to the survival of animals receiving an equal inoculum of wild-type bacteria (e.g., without a mutation). All animals have access to chow and water ad libitum throughout an experiment.

An exemplary bacterial inoculum is prepared as follows. Enterococcus faecalis OG1RF or Enterococcal mutants are grown overnight in BHI broth at 37° C. with gentle shaking. The cells are harvested by centrifugation, washed once with 0.9% saline, and then are resuspended in saline to an optical density of 2.2 to 2.8 at 600 nm. CFUs (colony-forming units) of cells suspensions are determined by plating serial dilutions onto BHI agar plates. Serial dilutions are prepared in saline and mixed with SRFE to the desired inoculum. For the preparation of SRFE, rat feces are dried, crushed, mixed with a volume of sterile distilled water three times that of the feces, and autoclaved. The resultant slurry is centrifuged, and the fecal extracts are removed aseptically. The extracts are then autoclaved and mixed with an enterococcal culture. Each inoculum is then diluted to a final 35% SRFE to yield the desired final inoculum.

Using a 25-gauge needle, mice are injected intraperitoneally with a 1 ml inoculum containing approximately 5×10⁸ to 1×10⁹ colony forming units of E. faecalis or an Enterococcal mutant. After injection the animals are returned to their cages and monitored every 8 hours for seven days. Surviving animals are then sacrificed and examined by autopsy. Control mice injected intrperitoneally with 1 ml of sterile SRFE are also examined.

Upon autopsy, bacteria are recovered from the kidneys or spleens under aseptic conditions. Peritoneal fluid and abdominal abscesses are also sampled for evaluation. Serial dilutions of the peritoneal fluid are prepared and 0.1 ml of each dilution is spread on agar plates for colony counts. Plates are then incubated under aerobic conditions for up to 4 days. BHI plates containing rifampin (for culturing Enterococcal wild type Enterococcus OG1RF) or rifampin and erythromycin (for culturing Enterococcal mutants) are used for selection. Results are expressed, for example, by Kaplan-Meier curves and log rank test using STATA software (StataCorp. 1999. Stata Statistical Software: Release 6.0. College Station, Tex.: Stata Corporation).

Mutants showing a statistically significant difference or a statistical trend (P≦0.20) compared to the wild type are, if desired, evaluated a second time. Mutants identified as having reduced virulence are taken as being useful in the invention.

### Compound Screening Assays

As discussed above, our experimental results demonstrated that Enterococcal virulence factors are involved in pathogenicity of the nematode, C. elegans. Based on this discovery we have also developed a screening procedure for identifying therapeutic compounds (e.g., anti-pathogenicity pharmaceuticals) which can be used to inhibit the ability of the Enterococcal pathogen to cause infection. In general, the method involves screening any number of compounds for therapeutically-active agents by employing the Enterococcal/nematode killing system described herein. Based on our demonstration that these pathogens infect and kill C. elegans, it will be readily understood that a compound which interferes with the pathogenicity of Enterococcus in a nematode also provides an effective therapeutic agent in a mammal (e.g., a human patient). Whereas most antibiotics currently in medical use are either bactericidal or bacteriostatic, thus favoring resistant strains or mutants, the compounds identified in the screening procedures described herein do not kill the bacteria but instead render them non-pathogenic. Moreover, since the screening procedures of the invention are performed in vivo, it is also unlikely that the identified compounds will be highly toxic to the host organism.

Accordingly, the methods of the invention simplify the evaluation, identification, and development of active agents such as drugs for the treatment of pathogenic diseases caused by Enterococcal microbes.

In general, the chemical screening methods of the invention provide a straightforward means for selecting natural product extracts or compounds of interest from a large population which are further evaluated and condensed to a few active and selective materials. Constituents of this pool are then purified and evaluated in the methods of the invention to determine their anti-pathogenic activity.

### Test Extracts and Compounds

In general, novel anti-pathogenic drugs are identified from large libraries of both natural product or synthetic (or semi-synthetic) extracts or chemical libraries according to methods known in the art. The screening method of the present invention is appropriate and useful for testing compounds from a variety of sources for possible anti-pathogenic activity. The initial screens may be performed using a diverse library of compounds, but the method is suitable for a variety of other compounds and compound libraries. Such compound libraries can be combinatorial libraries, natural product libraries, or other small molecule libraries. In addition, compounds from commercial sources can be tested, as well as commercially available analogs of identified inhibitors.

For example, those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-pathogenic activity should be employed whenever possible.

When a crude extract is found to have anti-pathogenic activity, further fractionation of the positive lead extract is necessary to isolate chemical constituents responsible for the observed effect. Thus, the goal of the extraction, fractionation, and purification process is the careful characterization and identification of a chemical entity within the crude extract having anti-pathogenic activity. Methods of fractionation and purification of such heterogenous extracts are known in the art. If desired, compounds shown to be useful agents for the treatment of pathogenicity are chemically modified according to methods known in the art.

Since many of the compounds in libraries such as combinatorial and natural products libraries, as well as in natural products preparations, are not characterized, the screening methods of this invention provide novel compounds which are active as inhibitors or inducers in the particular screens, in addition to identifying known compounds which are active in the screens. Therefore, this invention includes such novel compounds, as well as the use of both novel and known compounds in pharmaceutical compositions and methods of treating.

### Exemplary High Throughput Screening Systems

To evaluate the efficacy of a molecule or compound in promoting host resistance to, or inhibiting pathogenicity of, Enterococcus, a number of high throughput assays may be utilized.

For example, to enable mass screening of large quantities of natural products, extracts, or compounds in an efficient and systematic fashion, Caenorhabditis elegans, (e.g., L4 hermaphrodite larvae or a mutant worm such as aex-2, unc-25, phm-2, eat-14, pgp-4, or mrp-1), are cultured in wells of a microtiter plate, facilitating the semiautomation of manipulations and full automation of data collection. As is discussed above, E. faecalis infects and kills C. elegans. If E. faecalis has diminished pathogenicity, then L4 worms live, develop into adult hermaphrodites, and produce thousands of live progeny. Accordingly, if C. elegans is incubated with the pathogen, the worms will die, unless a compound is present to reduce E. faecalis pathogenicity. The presence of such live progeny is easily detected using a variety of methods, including visual screening with standard microscopes.

To evaluate the ability of a test compound or extract to promote a host's resistance to a pathogen or to repress pathogenicity of a pathogen, a test compound or extract is inoculated at an appropriate dosage into an appropriate agar medium (e.g., BHI or M17 (Difco)) seeded with an appropriate amount of an overnight culture of a pathogen, e.g., E. faecalis. If desired, various concentrations of the test compound or extract can be inoculated to assess dosage effect on both the host and the pathogen. Control wells are inoculated with non-pathogenic bacteria (negative control) or a pathogen in the absence of a test compound or extract (positive control). Plates are then incubated 24 hours at 37° C. to facilitate the growth of the pathogen. Microtiter dishes are subsequently cooled to 25° C., and two C. elegans L4 hermaphrodite larva are added to the plate and incubated at 25° C., the upper limit for normal physiological integrity of C. elegans. At an appropriate time interval, e.g., one hundred to two hundred hours, wells are examined for surviving worms, the presence of progeny, or both, e.g., by visual screening or monitoring motion of worms using a motion detector.

In another working example, Enterococcus-mediated killing of C. elegans is carried out as follows. Brain heart infusion (BHI) agar medium (Difco) is autoclaved and poured into 35 mm tissue culture plates (Fisher). Appropriate antibiotics are added to the medium before pouring to prevent growth of E. coli, but allow for the growth of the particular Enterococcus strains being tested. A test compound or compound library is also added to the medium. On the tissue culture plate, 2 ml of BHI is inoculated with a single colony of the appropriate strain, grown at 37° C. for 4 to 5 hours, and 10 μl of the culture is plated on each plate. The plates are incubated at 37° C. overnight, and then brought to room temperature for 2 to 5 hours. Thirty C. elegans, at the L4 larval stage, are then placed on the lawn from a plate of OP50 E. coli. The plates are incubated at 25° C., and the number of worms found dead compared to the total number of plated worms are then counted at approximately 24 hour intervals. Each experimental condition is done in triplicate and repeated at least twice. At an appropriate time interval plates are examined for surviving worms.

Comparative studies between treated and control worms (or larvae) are used to determine the relative efficacy of the test molecule or compound in promoting the host's resistance to the pathogen or inhibiting the virulence of the pathogen. A test compound which effectively stimulates, boosts, enhances, increases, or promotes the host's resistance to the pathogen or which inhibits, inactivates, suppresses, represses, or controls pathogenicity of the pathogen, and does not significantly adversely affect the normal physiology, reproduction, or development of the worms is considered useful in the invention.

### Use

The methods of the invention provide a simple means for identifying Enterococcal virulence factors and compounds capable of either inhibiting pathogenicity or enhancing an organism's resistance capabilities to such pathogens. Accordingly, a chemical entity discovered to have medicinal value using the methods described herein are useful as either drugs, or as information for structural modification of existing anti-pathogenic compounds, e.g., by rational drug design.

For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections which provide continuous, sustained levels of the drug in the patient. Treatment of human patients or other animals will be carried out using a therapeutically effective amount of an anti-pathogenic agent in a physiologically-acceptable carrier. In the context of treating a bacterial infection a “therapeutically effective amount” or “pharmaceutically effective amount” indicates an amount of an antibacterial agent, e.g., as disclosed for this invention, which has a therapeutic effect. This generally refers to the inhibition, to some extent, of the normal cellular functioning of bacterial cells (e.g., Enterococcal cells) causing or contributing to a bacterial infection. The dose of antibacterial agent which is useful as a treatment is a “therapeutically effective amount.” Thus, as used herein, a therapeutically effective amount means an amount of an antibacterial agent which produces the desired therapeutic effect as judged by clinical trial results, standard animal models of infection, or both. This amount can be routinely determined by one skilled in the art and will vary depending upon several factors, such as the particular bacterial strain involved and the particular antibacterial agent used. This amount can further depend on the patient's height, weight, sex, age, and renal and liver function or other medical history. For these purposes, a therapeutic effect is one which relieves to some extent one or more of the symptoms of the infection and includes curing an infection.

The compositions containing antibacterial agents of virulence factors or genes can be administered for prophylactic or therapeutic treatments, or both. In therapeutic applications, the compositions are administered to a patient already suffering from an infection from bacteria (similarly for infections by other microbes), in an amount sufficient to cure or at least partially arrest the symptoms of the infection. An amount adequate to accomplish this is defined as “therapeutically effective amount.” Amounts effective for this use will depend on the severity and course of the infection, previous therapy, the patient's health status and response to the drugs, and the judgment of the treating physician. In prophylactic applications, compositions containing the compounds of the invention are administered to a patient susceptible to, or otherwise at risk of, a particular infection. Such an amount is defined to be a “prophylactically effective amount.” In this use, the precise amounts again depend on the patient's state of health, weight, and the like. However, generally, a suitable effective dose will be in the range of 0.1 to 10000 milligrams (mg) per recipient per day, preferably in the range of 10-5000 mg per day. The desired dosage is preferably presented in one, two, three, four, or more subdoses administered at appropriate intervals throughout the day. These subdoses can be administered as unit dosage forms, for example, containing 5 to 1000 mg, preferably 10 to 100 mg of active ingredient per unit dosage form. Preferably, the compounds of the invention will be administered in amounts of between about 2.0 mg/kg to 25 mg/kg of patient body weight, between about one to four times per day.

Suitable carriers and their formulation are described, for example, in Remington's Pharmaceutical Sciences by E. W. Martin. The amount of the anti-pathogenic agent to be administered varies depending upon the manner of administration, the age and body weight of the patient, and with the type of disease and extensiveness of the disease. Generally, amounts will be in the range of those used for other agents used in the treatment of other microbial diseases, although in certain instances lower amounts will be needed because of the increased specificity of the compound. A compound is administered at a dosage that inhibits microbial proliferation.

All publications and patents mentioned in this specification are herein incorporated by reference to the same extent as if each individual publication or patent was specifically and individually indicated to be incorporated by reference.

From the foregoing description, one skilled in the art can easily ascertain the essential characteristics of this invention, can make various changes and modifications of the invention to adapt it to various usages and conditions. Thus, other embodiments are also within the claims.

What is claimed is:

1. A method for identifying an Enterococcal virulence factor, comprising the steps of:
(a) exposing a nematode to a mutagenized Enterococcal pathogen;
(b) determining whether said Enterococcal mutant infects said nematode, a reduction of disease in said nematode relative to that caused by the non-mutagenized Enterococcal pathogen indicating a mutation in an Enterococcal virulence factor; and
(c) using said mutation as a marker for identifying said Enterococcal virulence factor.

2. The method of claim 1, wherein said Enterococcal pathogen is Enterococcus faecalis.

3. The method of claim 1, wherein said Enterococcal pathogen is Enterococcus faecalis strain V583.

4. The method of claim 1, wherein said nematode is Caenorhabditis elegans.

5. The method of claim 1, wherein said method utilizes an Enterococcal/C. elegans killing assay.

6. The method of claim 5, wherein said mutated Enterococcal pathogen causes less C. elegans killing than the non-mutagenized Enterococcal pathogen.

7. A method of identifying a compound that inhibits pathogenicity of an Enterococcal pathogen, comprising the steps of:
(a) providing a nematode comprising an Enterococcal pathogen;
(b) contacting said nematode with a test compound; and
(c) determining whether the test compound inhibits the pathogenicity of said Enterococcal pathogen in said nematode.

8. The method of claim 7, wherein said Enterococcal pathogen is Enterococcus faecalis.

9. The method of claim 7, wherein said Enterococcal pathogen is Enterococcus faecalis strain V583.

10. The method of claim 7, wherein said nematode is Caenorhabditis elegans.

11. The method of claim 7, wherein said test compound is provided in a compound library.

12. The method of claim 7, wherein said test compound is a small organic compound.

13. The method of claim 7, wherein said test compound is a peptide, peptidomimetic, or antibody or fragment thereof.

14. The method of claim 7, wherein said inhibition of pathogenicity is measured by an Enterococcal/C. elegans killing assay.

15. The method of claim 14, wherein said Enterococcal pathogen causes less C. elegans killing in the presence of said test compound than in the absence of said test compound.

16. An isolated nematode comprising an isolated Enterococcal pathogen.

17. The nematode of claim 16, wherein said nematode is a mutated nematode.

18. The nematode of claim 16, wherein said nematode is Caenorhabditis elegans.

19. The nematode of claim 16, wherein said Enterococcal pathogen is a mutated pathogen.

20. The nematode of claim 16, wherein said Enterococcal pathogen is Enterococcus faecalis.

21. The nematode of claim 16, wherein said Enterococcal pathogen is Enterococcus faecalis strain V583.
